(19) United States
(12) Patent Application Publication
(10) Pub. No.: US 2025/0345141 A1
(43) Pub. Date: Nov. 13, 2025

(54) MEDICAL IMAGING COMPATIBLE RADIOLUCENT ACTUATION

(71) Applicant: Michael Campagna, Tampa, FL (US)

(72) Inventor: Michael Campagna, Tampa, FL (US)

(21) Appl. No.: 18/660,233

(22) Filed: May 10, 2024

(51) Int. Cl. A61B 34/00; A61B 34/37

(52) U.S. Cl. CPC A61B 34/76; A61B 34/37; A61B 2034/741

(57) ABSTRACT

A radiolucent medical imaging compatible approach of gathering stress and strain data from radiolucent pressure sensors arrayed along radiolucent surgical robotic circumduction end effectors, of transmitting electrical current and said sensor data via non-metallic yet conductive means, and of providing this sensor data as real-time haptic feedback to a surgeon's hand, fingers, thumbs and wrist via naturalistic control glove which issues gestural commands to the radiolucent end effector, for purposes of performing minimally-invasive robotic surgery, dissection, retraction, electrocautery, anastomosis and for delivering collision avoidance of said end-effectors within the radiographic and magnetic imaging bores.

### RELATED PATENTS

This application U.S. Divisional patent application Ser. No. 18/388,856 filed on Nov. 12, 2023, that claimed priority to U.S. patent application Ser. No. 16/908,620 filed on Jun. 22, 2020, that claims priority to U.S. Provisional Patent Application No. 63/038,743, titled “MEDICAL IMAGING COMPATIBLE RADIOLUCENT ACTUATION OF TRANSLATION ROTATION ARTICULATION CIRCUMDUCTION,” filed on Jun. 12, 2020, by Michael Campagna. The above applications are incorporated by reference only, and no claim to priority is made.

### TECHNICAL FIELD

The present invention relates generally to robotics, surgical robotics and to radiolucent medical sensors.

### BACKGROUND

The present state of the art in minimally-invasive surgical robotics is characterized by surgical robotic end effectors, which are primarily metallic in composition and utilize metallic compositions as the means of electrical transmission of current and data and are thereby inaccessible to usage within radiographic and magnetic imaging bores and sensors due to the incidence of high density and high attenuation artifact as well as the MRI “Black Hole” artifact occurring do to metal usage in the surgical robotics end effectors.

As such, the current surgical robots are unable to remain within the Live MRI/CT Imaging Bores and, therefore, unable to interface with the three-dimensional magnetic and radiographic, MRI & CT (3D) image-guided surgery, and is ONLY able to utilize low resolution photo-acoustic 3D image guidance which primarily identifies blood vessels as opposed to soft tissues.

When surgical robotics is UNABLE to function in an MRI & CT 3D image-guided surgery (IGS), a real problem exists in the art.

Furthermore, current surgical robotics control systems is fundamentally non-naturalistic and non-intuitive robotic joints as it Exaggerates as opposed to replicates the actual motions performed by of the surgeon, thereby introducing a learning curve, which ALSO represents a very real problem.

What is needed in the art is an approach to overcome the problems identified in the art.

### SUMMARY

The method and apparatus of Micra-Reach discloses a means to enhance the surgeon's direct visceral, tactile and naturalistic control experience and presents a radiolucent medical imaging compatible means of gathering stress, strain, and pressure data from radiolucent sensors arrayed along radiolucent surgical robotic circumduction end effectors, of transmitting electrical current and said sensor data via non-metallic yet conductive means, and of providing this sensor data as real-time haptic feedback to a surgeon's hands, fingers, thumbs, and wrists via naturalistic control glove which issues gestural commands to the radiolucent end effector, for purposes of performing minimally-invasive robotic surgery, dissection, retraction, electrocautery, anastomosis, and for delivering collision avoidance of said end-effectors, all of the above operable within radiographic and magnetic imaging bores.

Enabled via the usage of carbon based strain gauge pressure sensors and carbon based stress gauge haptic pressure sensors arrayed along the working surfaces of the radiolucent circumduction end effector, via the usage of non-metallic conductive “Yttrium” stabilized or “Skandia” stabilized zirconium blades for electrocautery, and via the incorporation of a radiolucent non-metallic flexible graphene-cotton thread based, or non-metallic flexible silicone-carbon nanotube electrical transmission array, all of these said components may be configured in a radiolucent non-metallic medical imaging compatible manner for usage & incorporation within the radiolucent surgical robotics circumduction end effectors and rendered operable via a gestural control glove provided with piezo-electric haptic feedback actuators in the interior of the glove.

All radiolucent strain, stress and pressure gauge haptic sensors, all radiolucent electrocautery and electrical transmission array of the radiolucent robotic end effector are thereby enabled to function within the MRI, CT, O-arm and radiographic imaging bores in a non-metallic medically imaging compatible and radiolucent manner which neither significantly affects the quality of the diagnostic information nor has its operations affected by the medical imaging system.

In this manner the gestural haptic control glove of the present invention will provide the surgeon with immediate real-time visceral sensations to the surgeon's fingers, thumbs, hands, and wrists which will replicate the stress and strain encountered by the radiolucent end effector during the performance of surgery, this haptic feedback conveyed as the tactile experience of actual contact of the end effector tools with patient tissues. Via this said instantaneous communication of stress, strain and pressure feedback gathered at the control surfaces of the radiolucent circumduction end effectors, with these said electrical impulses then transmitted along the non-metallic yet conductive graphene thread and or non-metallic yet conductive silicone graphene nanotube comprised electrical array, and then these electrical impulses transformed via microprocessor instantaneously into corresponding sensations relayed to the surgeons fingers and thumbs and hands within the haptic gestural control gloves.

In this way, the surgeon will obtain the immediate visceral feel of such sensations via haptic feedback actuators within the glove corresponding to equivalent portions of the end effector, which will convey and deliver directly to the surgeon's fingers the immediate tactile experience of actual contact of the end effector tools with patient tissues, as well as conveying the corporeal feedback of the grasp function of passing needles during the performance of anastomosis, as well as conveying the haptic sense of pressure or resistance during the grip and release functions and scissoring functions performed with the end effectors during dissection, electro-cautery/coagulation and anastomosis, as well as to convey the impact of tool collisions for purposes of collision avoidance.

The above is enabled via equipping the radiolucent micra track circumduction wrist end effector with non-metallic, carbon-based, pressure sensors on the distal tip and the medial aspect grip surfaces which correspond to the pincer and scissor grasp surfaces of the radiolucent circumduction end effector for purposes of conveying the tactile sensations of the scissoring action and the pincer action which replicates the end effector function of usage of various forceps, drivers, retractors, clip appliers, and via equipping the superior, inferior, and lateral surfaces of the radiolucent end effector, for purposes of collision sensation and avoidance.

The present invention “Micra-Reach” is an acronym for, “Medically Imaging Compatible Radiolucent Actuation of Robotic Electrical Array, Cautery & Haptics,” which describes the true function of the present invention and which translates as medically imaging compatible radiolucent actuation of robotic electrical array, cautery & haptics.

In the exemplary embodiment, the radiolucent gestural control glove of the present invention may be utilized to control the radiolucent end effectors with either a scissoring action of the surgeon's forefinger and the middle finger, or may be utilized to control the radiolucent end effectors with a pincer action of the surgeon's thumb and forefinger. The surgeon may easily switch from the scissoring to the pincer means of gestural control of the grasp and release function via foot pedal operation, as well as switch from tool to tool via the same means. In this manner, the gestural haptic feedback control glove may utilize the naturalistic thumb and forefinger pincer motion to control the grasp and release functions of the radiolucent end effector in the performance of retraction and the performance of needle passing during anastomosis, while the gestural haptic feedback control glove may also utilize the naturalistic forefinger and middle finger scissoring function in the performance of the scissoring functions of dissection and electro-cautery, all in the most naturalistic way and performed with radiolucent end effectors which replicate the actual motion of the surgeons wrist and finger and thumb motions, with all real-time stress and strain data communicated instantaneously as haptic feedback from the radiolucent end effector to the corresponding finger and thumb control surfaces within the gestural control glove of the present invention, all enabled to be performed in a radiolucent medically imaging compatible manner within the magnetic and radiographic imaging bore, and compatible with the usage of emerging real-time 2D (Two Dimensional) & 3D (Three Dimensional) image guidance within these imaging bores.

Other devices, apparatus, systems, methods, features, and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale; emphasis is instead placed upon illustrating the principles of the invention. In the figures, like reference numerals designate corresponding parts throughout the different views.

FIG. 1 is diagrams 1A-1E of a sensor and gestural control glove arrayed with motion capture sensors in accordance with an example embodiment.

FIG. 2 is diagrams 2A-2B of gestural control glove arrayed with piezo-electric haptic/tactile feedback devices depicted further depicted in 2C-2D in accordance with an example embodiment.

FIG. 3 is a diagram of end effectors 3A-3B arrayed with Carbon-based 3C non-metallic piezo-electric sensors 3D-3F in accordance with an example embodiment.

FIG. 4 is a diagram of the blades 4A and wires 4B in the end effector in accordance with an example implementation.

FIG. 5 is a diagram 5A and 5B of a surgeon with gestural control gloves having fiducials and sensors, where the fiducials are detected in gestural podium to control a surgical robot's radiolucent end effectors.

FIG. 6 is a diagram of a surgeon with gestural control gloves using flex and pressor sensors and a VR headset to control a surgical robot with radiolucent end effectors in accordance with an example embodiment.

### DETAILED DESCRIPTION

In order to detail the enhancements of Micra-Reach, to the radiolucent circumduction end-effectors and gestural control glove, we must first describe the function of the radiolucent circumduction end effector and gestural control glove using optical fiducial markers. The gestural podium utilizes motion capture optical fiducial markers placed upon the exterior of the control glove in order to translate the surgeon's gestures and commands into equivalent motions performed by the radiolucent circumduction end effector in the manner of motion capture as employed in the film-making industry, such that, as the surgeon's hand moves, this movement is captured via motion capture sensors arrayed within the gestural podium, which then track the position of the fiducial markers. Via microprocessor, this motion capture data of the surgeon's gestural commands is then translated into the equivalent and simultaneous motion of the radiolucent robotic end effector.

Micra-Reach enhances the gestural glove control system with additions to the outer portion of the glove and with additions to the inner portion of the glove, with the outer portion of the control glove utilizing piezo-electric strain and flex sensors and thereby capturing the gestural motions of the surgeon's hands and relaying these commands to the end effectors, and with the inner portion of the control glove translating the pressure gauge and piezo-electric data communicated from the end effectors directly to the surgeon's hands and fingers as haptic feedback/tactile sensations, thereby enabling the surgeon's sense of touch with real-time information, with all of said haptic feedback actuators communicating directly to the skin of the surgeon's fingers and thumbs and hands, enabling all of the surgical procedure to be performed by the direct motion of the surgeon's own fingers, thumbs, wrists, and hands, in the same manner as traditional surgery. The Micra-Reach gestural haptic control glove can be silicone, textile, or any other material that breathes, is flexible, and fits snugly to the surgeon's hand. This said, the present invention Micra-Reach may continue to employ optical motion capture via the tracking of reflective fiducial markers within the gestural podium to be utilized in concert with the piezo-electric flex sensors of the present invention as a further means of motion capture, adding additional fault tolerances, or may discontinue usage of the motion capture in favor of sole usage of the piezo-electric flex sensor means of motion capture, depending on the option which is best suited to specific usage and the size of the gestural podium console.

The outer portion of the micra-trac gestural control glove is arrayed with piezo-electric flex sensors and piezo-electric strain sensors for motion capture of the surgeon's gestural commands. These highly flexible stain sensors and flex sensors translate the gestural and motion commands of the surgeon's finger's, thumbs, hands, and wrists directly to a computer processor, which converts these gestural motion commands instantaneously into either radiolucent cable actuation of the radiolucent circumduction end effector, or commands via solenoid manifold for pneumatic actuation of the end effector. A solenoid manifold may be configured as hydraulically actuated as opposed to magnetically actuated for purposes of usage with proximity of the MRI. (The present invention may also continue to utilize optical fiducial markers on the exterior of the gestural control gloves' surface for image capture purposes.)

Specifically, piezo-electric flex sensors and piezo-electric strain sensors are positioned along the superior, inferior, and lateral aspects of the thumb, forefinger, and middle finger, as well as upon the distal tips of these digits for purposes of enhanced motion capture of the surgeon's gestures and simultaneous translation via microprocessor into gestural motion commands. The gestural control glove of the present invention may be utilized to control the radiolucent end effectors with either a scissoring action of the surgeon's forefinger and the middle finger, or may be utilized to control the radiolucent end effectors with a pincer action of the surgeon's thumb and forefinger, and may switch between both of these modalities via foot pedal. Additionally, piezo-electric strain sensors are arrayed circumferentially around the superior, inferior, and lateral aspects of the wrist to capture the abduction/adduction, flexion/extension, translation, rotation, and circumduction functions of the surgeon's wrist for transformation of this flex data into gestural commands transmitted to the radiolucent end effector. The Micra-Reach gestural haptic control glove may also be enhanced via the lengthening of the glove to encompass the surgeon's elbow, or even the arm and shoulder as well, or via the addition of an elbow pad flex sensor arrayed with visual tracking fiducial markers for usage within the gestural podium, for purposes of capturing the articulation and translation of the surgeon's entire forearm, for purposes of remedying the “clutching” problem which is common to the current state of the art of surgical end-effectors, this said “clutching” being the necessity of the surgeon having to reset the current state of the art surgical end effectors after manipulation of the end effector at the distal reach of its limitations.

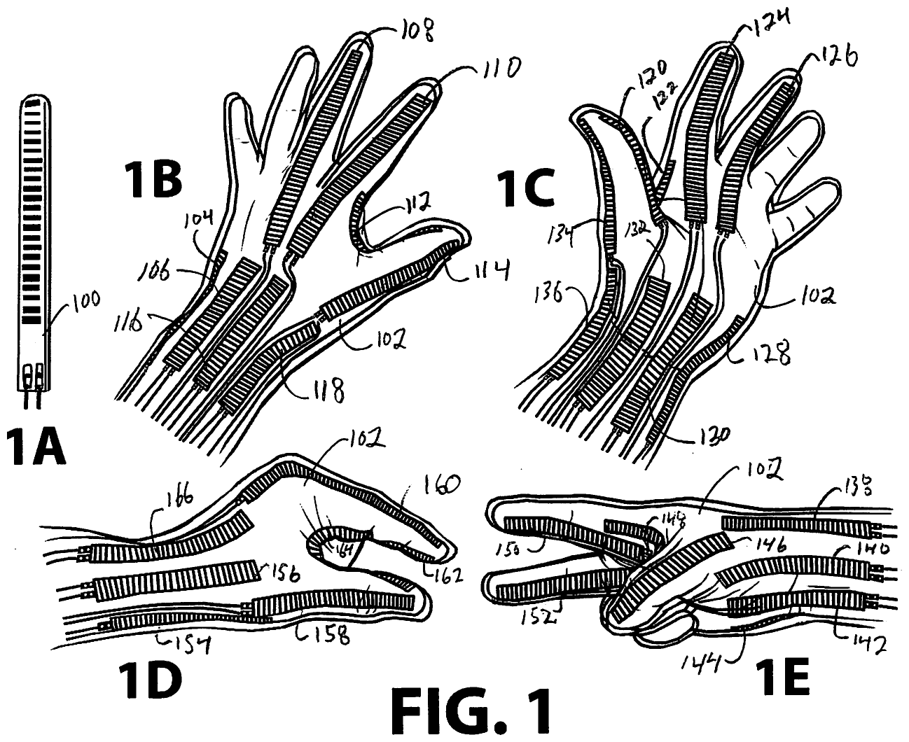

The inner portion of the gestural control glove 102 FIG. 1 is arrayed with ultra-thin piezo-electric haptic feedback actuators along the interior of the gestural control glove such that electrical signals originating from the stress and strain sensors arrayed within the radiolucent circumduction end-effectors may be transmitted directly to a computer processor, which converts this data instantaneously into visceral force feedback delivered to the surgeon's hands and fingers and thumbs via the Micra-Reach control glove's haptic piezo-electric feedback array for purposes of replication of the stress and strain encountered by the radiolucent end effector sensors during the performance of surgery. In this manner, haptic feedback is then conveyed instantaneously to the surgeon's hand, fingers, and thumb as the real-time tactile experience of actual contact of the end effector tools with patient tissues, as well as conveying corporeal feedback of the grasp function of passing needles during the performance of anastomosis, as well as conveying the haptic sense of pressure or resistance during the grip and release functions and retraction and dissection scissoring functions performed with the end effectors during surgery, as well as to convey the occurrence of tool collisions for purposes of collision avoidance, with all of the said haptic feedback within the naturalistic gestural haptic control glove 102 FIG. 1 corresponding to the equivalent portion of the end effector.

Specifically, piezo-electric haptic feedback actuators are positioned facing inward within the interior of the gestural control glove 102 FIG. 1 so as to be in contact along the superior, inferior, and lateral aspects of the surgeon's thumb, forefinger, and middle finger as well as upon the distal tips of these said digits, thereby corresponding to the grip and release portion of the radiolucent end effector. In this manner, the surgeon will obtain the immediate visceral feel of the pressure of the grasp and release functions of the radiolucent circumduction end-effector via haptic feedback within the glove 102 FIG. 1 corresponding to the equivalent portion of the end effector, as well as to convey to the surgeon's fingers the tactile experience of actual contact of the end effector tools with objects during the grip and release functions and scissoring functions performed with the end effectors during surgery. Furthermore, piezo-electric haptic feedback actuators are also positioned facing inward within the interior of the gestural control glove 102 FIG. 1 so as to be in contact with the superior and inferior aspects of the surgeon's hand, as well as along the lateral aspects of the palm and the “back of the hand” for purposes of collision avoidance of the multiple tools within the surgical space, such that the surgeon would be able to feel these said collisions within the haptic control glove 102 FIG. 1 as pressure along the aspects of the point of contact and is thereby enabled to avoid them.

The radiolucent circumduction end effectors are arrayed with carbon-based non-metallic imaging compatible and radiolucent stress strain and pressure sensors along the control surfaces and along points of possible collision contact, these said radiolucent carbon based stress and strain sensors fashioned from carbon materials i.e. CNT carbon nanotubes, graphene, carbon black, graphite or carbon silicone hybrids, carbon-polymer hybrids, solidified carbon ink, CNT carbon nano-tube (without limitation), any of these said materials then configured into textiles, fibers, films as strain and pressure sensors, these said strain and pressure sensors then employing non-metallic conductive Graphene impregnated cotton thread fabric or graphene-silicone wires as the means of electrical transmission of this real-time pressure and strain data from the surfaces of the radiolucent end effector for delivery as haptic feedback to the surgeons hand, fingers, thumbs and wrist within the haptic-feedback enabled gestural control glove 102 FIG. 1, as well as for purposes of sensing instrument collisions for purposes of collision avoidance. These radiolucent carbon-based piezo-electric stress and strain sensors and pressure sensors are positioned along the superior, inferior, and lateral aspects of the grasping portion of the radiolucent circumduction end effector as well as upon the distal tips, such that electrical signals originating from the stress and strain pressure sensors arrayed within the radiolucent circumduction end-effectors may be transmitted directly to a computer processor, which converts this data instantaneously into visceral force feedback delivered to the surgeon's hands and fingers and thumbs via the control glove's haptic piezo-electric feedback actuator array. In this manner, the surgeon will obtain the immediate visceral feel of the pressure of the grasp and release functions of the radiolucent circumduction end-effector via haptic feedback within the glove 102 FIG. 1 corresponding to the equivalent portion of the end effector, as well as to convey to the surgeon's fingers the tactile experience of actual contact of the end effector tools with patient tissues, as well as conveying corporeal feedback of the grasp function of passing needles during the performance of anastomosis, as well as conveying the haptic sense of pressure or resistance during the grip and release functions and scissoring functions performed with the end effectors during surgery. Furthermore, the superior and inferior aspects of the radiolucent end-effector, and both lateral aspects of the end effector, may be arrayed with ultra-thin carbon-based piezo-electric strain and pressure sensors for purposes of collision avoidance of the multiple tools within the surgical space, such that the surgeon would be able to feel these said collisions within the haptic control glove 102 FIG. 1 as pressure along the aspects of the point of contact, and is thereby enabled to avoid them. Finally, all of these said radiolucent piezo-electric strain gauges and pressure sensors may be rendered waterproof and thereby workable within the surgical site, as well as adhered to the surfaces of the end effector via the usage of RTV (room temperature vulcanized) silicone coatings, or by other equivalent radiolucent non-metallic means.

The radiolucent end-effectors are configured with non-metallic, non-magnetic, and thereby imaging compatible and radiolucent “Yttrium” stabilized Zirconium Dioxide (YSZ) blades or with “Skandia” stabilized Zirconium Dioxide (ScZ) blades, for purposes of delivering non-metallic imaging compatible and radiolucent bipolar electrocautery to the surgical site, which requires no metallic ground plate, and is thereby accessible to the MRI and CT imaging bores.

The radiolucent end-effectors are configured with non-metallic imaging compatible and radiolucent fiber optics for illumination and image capture. The end effector's grasp function may be arranged to provide a wiper for the lens to keep it clear of debris and obscuration during the performance of surgery. Live images captured via these fiber optics within either the endoscope and delivered from end effector fiber optics array can be viewed on the gestural podium imaging screen or via a heads-up display, optical helmet, glasses, or even via a holographic display.

In one embodiment, the radiolucent circumduction end-effectors are configured with non-metallic conductive cotton thread-based Graphene and, or Graphene Nanotube Silicone composites employed as non-metallic and medically imaging-compatible radiolucent wires for the transmission of electrical signals originating from the stress and strain sensors arrayed within the radiolucent circumduction end-effectors, these electrical signals then sent to the control glove's haptic piezo-electric feedback array for real-time micro-processor translation into visceral force feedback delivered to the surgeon's hands and fingers and thumbs. These non-metallic conductive cotton thread-based Graphene OR Graphene Nanotube Silicone composites, are also configured as wires for the transmission of electrical current to the non-metallic, imaging compatible, “Yttrium” Stabilized Zirconium (YSZ) bipolar forceps for purposes of powering the non-metallic bipolar electrocautery during the surgical procedure. All of these said non-metallic electrical signal wires and non-metallic electrical transmission “wires” are rendered waterproof via enclosure within flexible PVC and, or silicone tubing, all for purposes of delivering non-metallic imaging compatible and radiolucent electrical impulse and electrical current transmission array to the surgical site within the imaging environment. These said conductive thread-based Graphene impregnated transmission wires may also be configured from any other absorbent and flexible fabric-thread based materials in addition to cotton, to include without limitation, Rayon, Hemp, Linen, Silk, Wool, Bamboo Textiles, and other thread-based fabrics as may be amenable, as the fabric thread functions merely as the absorbent and flexible substrate, whereas the Graphene which has been impregnated into to this flexible substrate, is the actual means of non-metallic and radiolucent electrical conduction.

All of the above details are enabled to function within the magnetic and radiographic MRI, CT, & O-Arm imaging bores in a non-metallic medically imaging compatible and radiolucent manner, which neither significantly affects the quality of the diagnostic information nor has it's operations affected by the medical imaging system. In this manner, the surgeon is enabled to perform the most intricate micro-surgery with all of the skill of the surgeon's actual hands and digits reduced to the micro level, and yet with all of the real-time sensation and feedback and visualization conveyed as though the surgeon was seated directly within arm's reach of the microsurgical site, operating naturalistically with no learning curve involved, and with all of the benefit of the most advanced real-time 3D imaging guidance at their disposal.

In an exemplary embodiment, the following instruments generally work together in concert and comprise two radiolucent circumduction end-effectors w forceps, one radiolucent circumduction end-effector arrayed with zirconium dioxide bipolar dissection/electrocautery blades, and one radiolucent circumduction end effector arrayed with fiber-optics to deliver illumination and transmit visuals of the live surgical site, with the grasp-release portion of the end effector configured as lens wiper in order to keep the fiber-optic array free from occlusion intra-operatively.

An alternative embodiment of the Micra-Reach radiolucent end-effector with gestural haptic control glove 102 FIG. 1 may also be incorporated within radiolucent flexible surgical robotics platforms, via positioning of the radiolucent circumduction end effector at the distal tip of the flexible robot, with all non-metallic cotton thread-based graphene wires and, or Graphene silicone electrical conductivity wires, non-metallic cable actuation wires, and/or pneumatic lines configured to pass through a series of central foramina magna (plural of foramen magnus). In this way, the radiolucent circumduction end-effector with gestural haptic control glove 102 FIG. 1 may be further enabled to perform surgery within previously inaccessible sites.

In yet another alternative embodiment of the Micra-Reach radiolucent end-effector with gestural haptic control glove 102 FIG. 1, is a single port iteration of the present invention configured as a radiolucent circumduction end effector with radiolucent shoulder, radiolucent elbow, and radiolucent wrist, configured via the combination of the radiolucent three degrees of freedom circumduction joint and the radiolucent one degree of freedom flexion-extension joint. This iteration of a radiolucent Micra-Reach radiolucent shoulder, elbow, and wrist (SEW) end-effector (Micra-Reach sew radiolucent end-effector) is disclosed for purposes of enabling the distal triangulation of multiple Micra-Reach sew radiolucent end-effectors through one radiolucent uni-port cannula for single port access of minimally invasive surgical procedures. One example of such a minimally invasive medically imaging-compatible procedure would be the 3D CT/computerized tomography image-guided in-utero microsurgery to correct for fetal cardiac or Spina-Bifida Malformations, with surgical access to the womb enabled via a single radiolucent port. Via this single radiolucent port, multiple of the Micra-Reach radiolucent SEW end effectors may access the surgical site via distal triangulation, with surgeon gestural control enabled via optical fiducial makers and image capture of a sleeved garment arrayed with optical fiducial markers from the fingertips to the surgeon's shoulders, thereby enabling the surgeon to control, two of these said radiolucent Micra-Reach SEW end effectors at once and entirely via gestural motions of the surgeon's fingers, thumbs, hand, wrist, elbow and shoulder. In this iteration, the surgeon may remain comfortably seated within a chair with arm and shoulder rests, and control the radiolucent SEW end effector via the naturalistic motions of the shoulder, elbow, wrist, fingers, and thumb. For the most part, the surgeon's gestural control will involve the elbow, wrists, fingers, and thumb, with only occasional gestural control of the shoulder. A large screen may be employed for usage with 3D glasses, such that the surgeon may sit comfortably either before this large screen while performing surgery, or the surgeon may be equipped with a VR headset with a heads-up augmented reality display through which the surgeon may visualize not only the images captured by the fiber optics array but may also visualize the 3D image guided scan, as well as various combinations of both the optical and the image-guided scan superimposed atop one another.

To reiterate, in this manner, the surgeon is enabled to perform the most intricate micro-surgery with all of the skill of the surgeon's actual hands and digits reduced to the micro level, and yet with all of the real-time sensation feedback and visualization conveyed as though the surgeon was seated directly within arm's reach of the microsurgical site, operating naturalistically with no learning curve involved, and with all of the benefit of the most advanced CT and MRI realtime 3D imaging guidance at their disposal.

In FIG. 1, diagram 1A-1E of a sensor 100 and gestural control glove 102 arrayed with motion capture sensors 104-166 is depicted in accordance with an example embodiment. Gestural glove control system via the addition of flex sensors and strain sensors 104-166 to the outer portion of the gestural glove 102 for purposes of enhanced motion capture of the surgeon's gestural commands, in order to transmit said commands to the end effector for immediate replication and synchronization. Depending upon the implementations, it is noted that the flex and strain sensors may be inside, outside, or a combination of inside and outside of the gestural glove 102 as depicted in 1E.

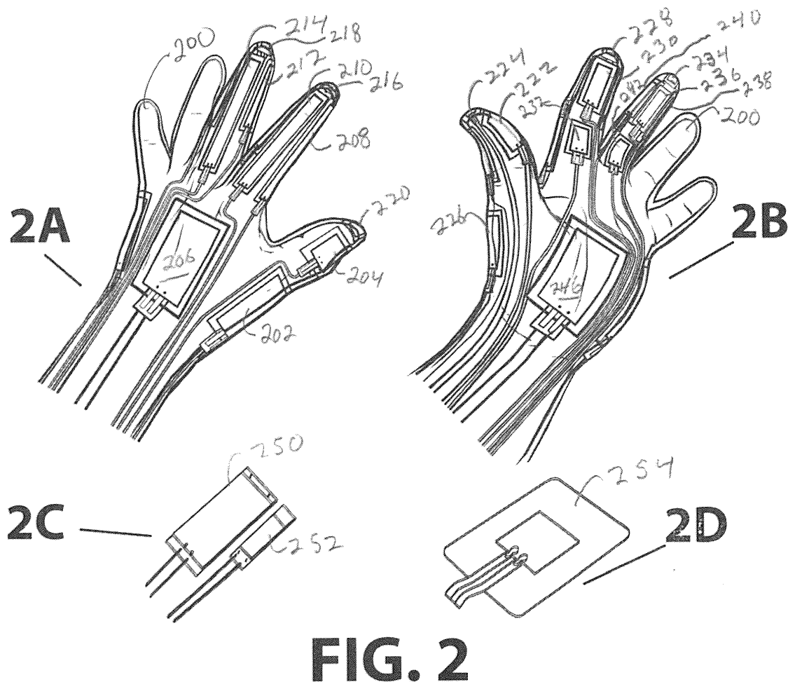

Turning to FIG. 2, diagrams 2A-2B of gestural control glove 200 arrayed with piezo-electric haptic/tactile feedback devices 202-248 depicted in 2C-2D 250-254 in accordance with an example embodiment. Micra-Reach enhances the surgeon's direct visceral experience of the gestural glove 200 control via the addition of piezo-electric haptic feedback delivered to the surgeon's fingers and hands by the piezo-electric haptic/tactile feedback devices 202-254 to convey the tactile sensations of the grip and release function of the end effector to the surgeon's hand, as well as strain gauges to provide resistance feedback to provide immediate haptic feedback for purposes of collision avoidance of the various surgical tools equipped with ultra-thin piezo-electric strain & resistance gauges within the operating field.

The feedback is enabled by equipping the radiolucent circumduction wrist end effector with non-metallic/piezo-electric crystal pressure sensors on the distal tip and the grip surfaces of a robot for purposes of conveying the tactile sensations of the scissoring action, which replicates the end effector function of usage of various forceps, drivers, retractors, and clip appliers. In this manner, the gestural haptic feedback control glove 200 may utilize the naturalistic thumb and forefinger pincer motion to control the grasp and release functions of the radiolucent end effector in the performance of retraction and the performance of needle passing during anastomosis, while the gestural haptic feedback control glove 200 may also utilize the naturalistic forefinger and middle finger scissoring function in the performance of the scissoring functions of dissection of the electrosurgical bipolar scissors for dissection and coagulation, electro-cautery, all in the most naturalistic way and performed with radiolucent end effectors which replicate the actual motion of the surgeons wrist and finger and thumb motions

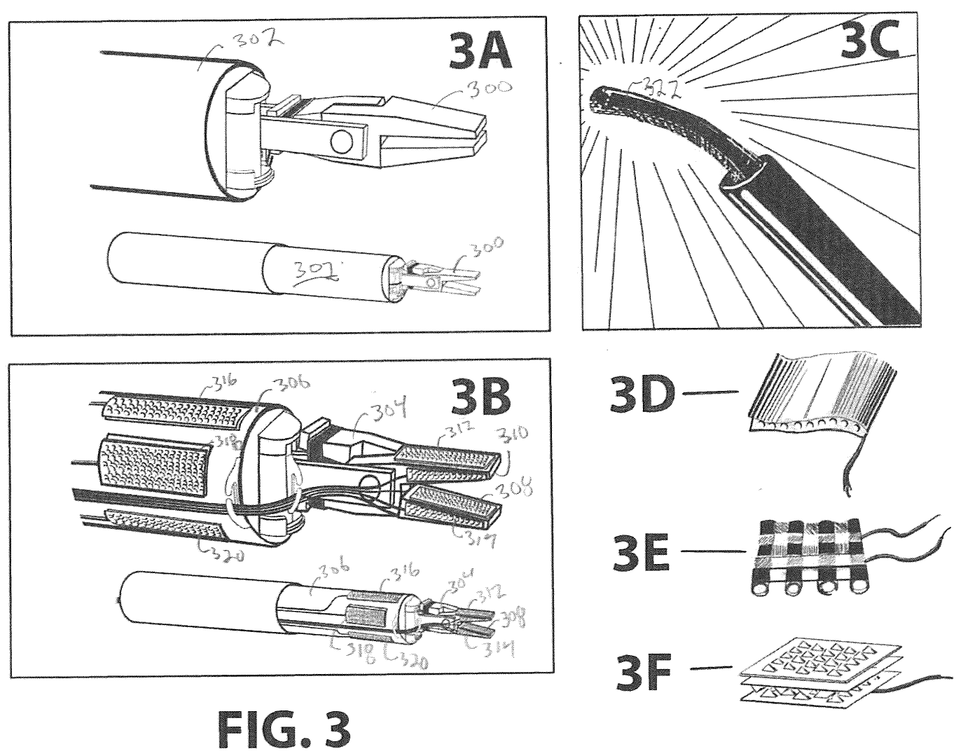

In FIG. 3, a diagram of end effectors 3A-3B arrayed with Carbon-based 3C non-metallic piezo-electric sensors 3D-3F is depicted in accordance with an example embodiment. An end effector shown in 3A has a gripping end 300 and rotatable/slidable portion 302. Micra-reach further enhances the surgeon's experience via the addition of carbon-based non-metallic piezo-electric strain gauges and crystal pressure sensors 308-320 on the distal tip of end effector 3B (304 and 306), within the medial aspect of the “V” portion, arrayed along inferior and lateral portions of the end effector 304, to affect collision avoidance, and utilizing, carbon-based “flex sensors” & “strain sensors” (fashioned from carbon nanotubes/graphene/carbon black/graphite, cordon thread based Graphene, solidified carbon ink) 3C with non-metallic conductive Graphene impregnated cotton thread fabric 322 or graphene-silicone wires as the means of electrical transmission of this real-time pressure and flex bend data to the control glove equipped with piezo-electric haptic feedback. All of the above are arrayed such that the surgeon can “feel” the real-time pressure and resistance of the grip and release function of the end effector, as well as the flex and bend within the glove upon their hand, as well as collisions. In this manner, the surgeon will obtain the immediate visceral feel of such tool collisions via haptic feedback within the glove corresponding to the equivalent portion of the end effector, as well as to convey to the surgeon's fingers the tactile experience of actual contact of the end effector tools with patient tissues, as well as conveying corporeal feedback of the grasp function of passing needles during the performance of anastomosis, as well as conveying the haptic sense of pressure or resistance during the grip and release functions and scissoring functions performed with the end effectors during surgery.

Furthermore, the superior aspect and both lateral aspects of the end effector may be arrayed with ultra-thin piezo-electric strain sensors for purposes of collision avoidance of the multiple tools within the surgical space, such that the surgeon would be able to feel these said collisions within the haptic control glove as pressure along the aspects of the point of contact and is thereby enabled to avoid them. Such sensors are silicone graphene pressure sensor 3D, Silicone Carbon nanotube 3E. All of these said piezo-electric strain gauges and pressure sensors may be rendered waterproof and thereby workable within the surgical site, as well as adhered to the surfaces of the end effector via the usage of RTV silicone (room temperature vulcanized) coatings as shown in 3F. In some embodiments, the pressure sensors may be transparent.

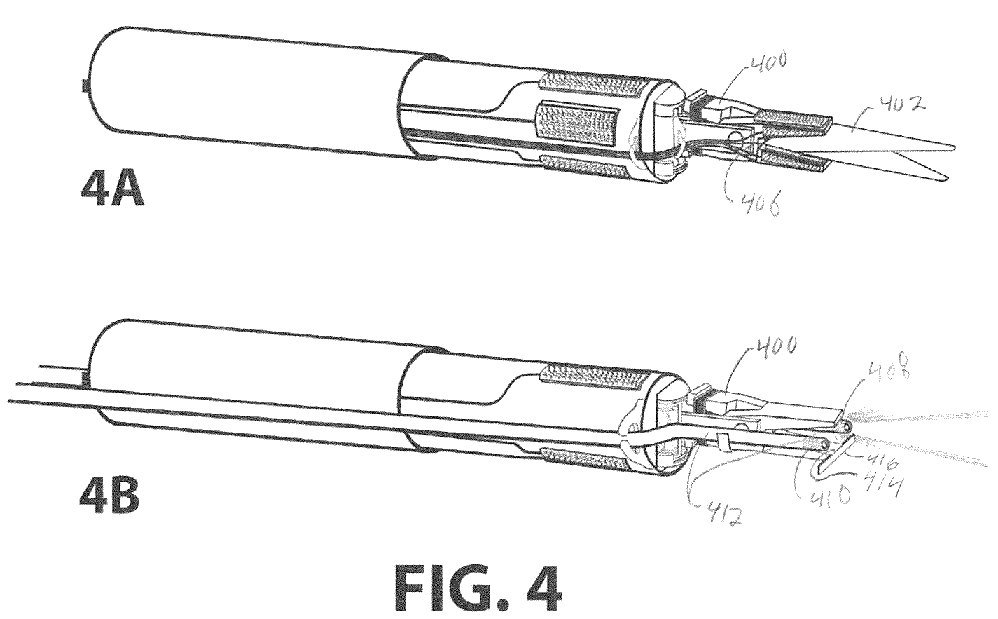

Turning to FIG. 4 is a diagram of the blades 4A and wires 4B in the end effector 400 is depicted in accordance with an example implementation. Non-metallic Yttriia stabilized Zirconium Dioxide (YSZ) bipolar electrocautery blades 402 are depicted. The usage of non-metallic electrically conductive Yttrium Stabilized Zirconium Dioxide for the delivery of bipolar electrocautery blade 402, with the end effector 400 as the articulating radiolucent armature to enable function of the Non-Metallic Bipolar Zirconium Bladed 400 Coagulation Forceps. Bipolar Electrocautery/Coagulation requires NO metal ground platform for the patient to be positioned upon (unlike mono-polar cautery and coagulation), thereby rendering this non-metallic approach accessible and usable within the MRI and CTI and O-Arm imaging bores. Non-metallic electrically conductive Scandia-Stabilized Zirconium (ScZC) Yttrium Stabilized Zirconium Dioxide for providing medically imaging compatible electrocautery & coagulation may also be incorporated.

Non-metallic conductive cotton thread-based graphene 406 for transmission of electric current to the non-metallic bipolar electrocautery and for the transmission of signals from the piezo-electric stain gauges and sensors arrayed in the end effectors to the surgeon's gestural control glove is depicted in the current embodiment. Furthermore, the usage of radiolucent, medically imaging compatible, non-metallic conductive graphene thread 406 and or Silicone Graphene Nanotubes, as the means of electrical current transmission means of electrical current transmission may be enclosed within flexible and radiolucent PVC and or silicone tubing for purposes of waterproofing and thereby rendered accessible and fully operable within the surgical site.

Non-Metallic Fiber-Optics Illumination 408 and Image Capture 410 embodiment is depicted in 4B. The usage of a radiolucent end effector 400 as the means of delivery of non-metallic fiber optics 412 to the surgical site, as both the means of illumination 408 and image transmission of the live procedure (a lesser quantity of fiber optics can also be utilized near the distal tip of the end effectors themselves to provide simple visual proximity data to aid in Collision Prevention of the various end effectors.) Additionally, the grasp function of the end effector 414, which houses the fiber optics illumination and image transmission cables and lens, may be configured to be utilized as a lens wiper 416 for cleaning the lens to keep the lens free from debris which may obscure visualization of the surgical site.

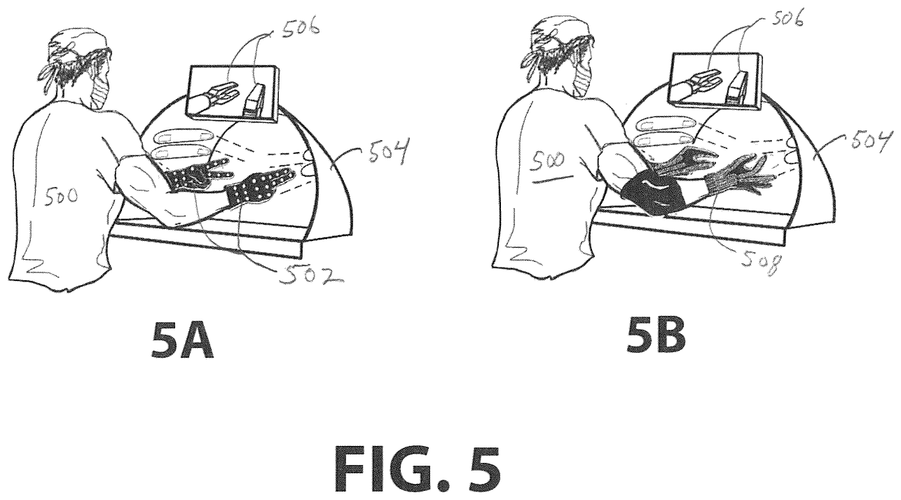

In FIG. 5, diagrams 5A and 5B of a surgeon 500 with gestural control gloves 502 having fiducials and sensors 508, where the fiducials are detected in gestural podium 504 to control a surgical robot's radiolucent end effectors in accordance with an example embodiment.

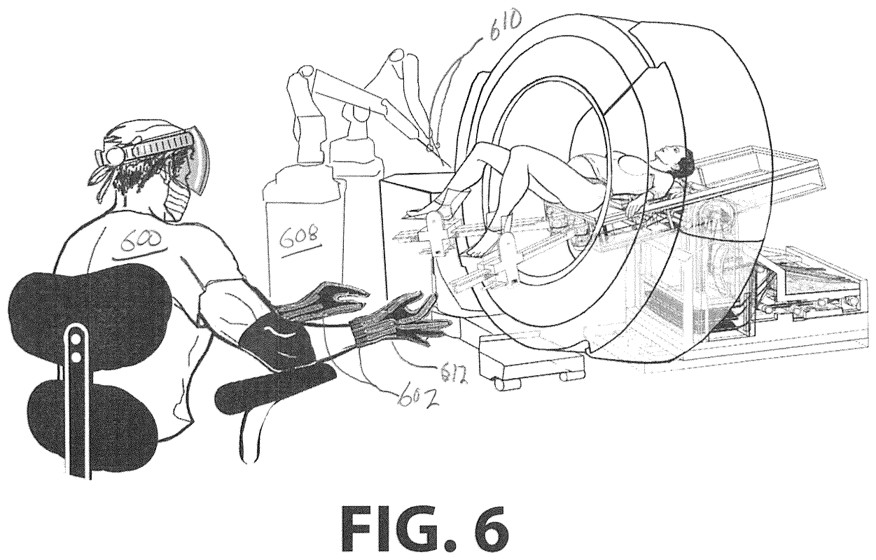

Turning to FIG. 6, a diagram of a surgeon 600 with gestural control gloves 602 using flex and pressor sensors 612 and a VR headset 604 to control a surgical robot 508 having radiolucent end effectors 610 is depicted in accordance with an example embodiment. The gestural control gloves 602 provide haptic feedback delivered to the surgeon 500 hands and fingers with piezo-electric haptic feedback. The VR headset 604 displays images from the surgical site via fiber optics. In other implementations, the VR headset 604 may be an augmented reality headset.

It will be understood and appreciated by persons skilled in the art, that one or more processes, sub-processes, or process steps may be performed by hardware and/or software (machine-readable instructions). If the approach is performed by software, the software may reside in software memory in a suitable electronic processing component or system such as one or more of the functional components or modules schematically depicted in the figures.

The software in software memory may include an ordered listing of executable instructions for implementing logical functions (that is, “logic” that may be implemented either in digital form, such as digital circuitry or source code, or in analog form, such as analog circuitry or an analog source such an analog electrical, sound or video signal), and may selectively be embodied in any computer-readable medium for use by or in connection with an instruction execution system, apparatus, or device, such as a computer-based system, processor-containing system, or other systems that may selectively fetch the instructions from the instruction execution system, apparatus, or device and execute the instructions. In the context of this disclosure, a “computer-readable medium” is any tangible means that may contain or store the program for use by or in connection with the instruction execution system, apparatus, or device. The tangible computer-readable medium may selectively be, for example, but is not limited to, an electronic, magnetic, optical, electromagnetic, or semiconductor system, apparatus, or device. More specific examples, but nonetheless a non-exhaustive list, of tangible computer-readable media would include the following: a portable computer diskette (magnetic), a RAM (electronic), a read-only memory “ROM” (electronic), an erasable programmable read-only memory (EPROM or Flash memory) (electronic) and a portable compact disc read-only memory “CDROM” (optical). Note that the tangible computer-readable medium may even be paper (punch cards or punch tape) or another suitable medium upon which the instructions may be electronically captured, then compiled, interpreted or otherwise processed in a suitable manner if necessary, and stored in a computer memory.

The foregoing detailed description of one or more embodiments of the approach for middleware service for integrated building server that communicates directly with equipment, panels, and points has been presented herein by way of example only and not limitation. It will be recognized that there are advantages to certain individual features and functions described herein that may be obtained without incorporating other features and functions described herein. Moreover, it will be recognized that various alternatives, modifications, variations, or improvements of the above-disclosed embodiments and other features and functions, or alternatives thereof, may be desirably combined into many other different embodiments, systems or applications. Presently unforeseen or unanticipated alternatives, modifications, variations, or improvements therein may be subsequently made by those skilled in the art which are also intended to be encompassed by the appended claims. Therefore, the spirit and scope of any appended claims should not be limited to the description of the embodiments contained herein.

What is claimed is:

1. A surgical robot, comprising:
a robotic base,
an at least one end effector coupled to the base, where the end effector is radiolucent,
an at least one radiolucent sensor coupled to the end effector that generates haptic feedback data, and
at least one radiolucent lead coupled to the radiolucent sensor that carries the feedback data.

2. The surgical robot of claim 1, where the at least one radiolucent sensor is a strain sensor.

3. The surgical robot of claim 2, where the at least one radiolucent sensor is a flex sensor.

4. The surgical robot of claim 1, wherein the at least one radiolucent lead is formed at least in part with Carbon nanotubes.

5. The surgical robot of claim 1, wherein the at least one radiolucent lead is formed at least in part with graphene.

6. The surgical robot of claim 5, where the at least one radiolucent lead formed at least in part with Graphene, includes cotton thread-based Graphene.

7. The surgical robot of claim 1, includes at least one glove that controls the end effector and receives the haptic feedback data via the at least one radiolucent lead, and
converts the haptic feedback data in a physical sensation at the glove.

8. A surgical robot method, comprising:
coupling an at least one end effector with a robotic base, where the end effector is radiolucent,
generating haptic feedback data with an at least one radiolucent sensor coupled to the end effector, and
carrying feedback data with the at least one radiolucent lead coupled to the radiolucent sensor.

9. The surgical robot of claim 8, where the at least one radiolucent sensor is a strain sensor.

10. The surgical robot of claim 8, where the at least one radiolucent sensor is a flex sensor.

11. The surgical robot of claim 8, wherein the at least one radiolucent lead is formed at least in part with Carbon nanotubes.

12. The surgical robot of claim 8, wherein the at least one radiolucent lead is formed at least in part with graphene.

13. The surgical robot of claim 12, where the at least one radiolucent lead formed at least in part with Graphene, includes cotton thread-based Graphene.

14. The surgical robot of claim 8, includes controlling the end effector with at least one glove,
receiving the haptic feedback data via the at least one radiolucent lead, and
converting the haptic feedback data into a physical sensation at the glove.
